SEQUENCE LISTING (12 sequences)

SEQ ID NO: 1 - DNA, 20 bp, Artificial Sequence
  synthetic oligonucleotide primer
gggctgcttttaactctggt

SEQ ID NO: 2 - DNA, 20 bp, Artificial Sequence
  synthetic oligonucleotide primer
tggcaggtttttctagacgg

SEQ ID NO: 3 - DNA, 20 bp, Artificial Sequence
  synthetic oligonucleotide primer
tttcccaggtcaagatggtc

SEQ ID NO: 4 - DNA, 20 bp, Artificial Sequence
  synthetic oligonucleotide primer
cttcagcacctgtctcacca

SEQ ID NO: 5 - DNA, 21 bp, Artificial Sequence
  synthetic oligonucleotide primer
gggaaaatgaagacctgctgg

SEQ ID NO: 6 - DNA, 23 bp, Artificial Sequence
  synthetic oligonucleotide primer
cgaaaaggctgctgtttggagac

SEQ ID NO: 7 - DNA, 24 bp, Artificial Sequence
  synthetic oligonucleotide primer
gcccaagaggtgcccctggaatac

SEQ ID NO: 8 - DNA, 24 bp, Artificial Sequence
  synthetic oligonucleotide primer
cctgagaaagaggagtggacatac

SEQ ID NO: 9 - DNA, 22 bp, Artificial Sequence
  synthetic oligonucleotide primer
ggaaaggacgaagttggtctgc

SEQ ID NO: 10 - DNA, 22 bp, Artificial Sequence
  synthetic oligonucleotide primer
cttctccacgctgattgctacc

SEQ ID NO: 11 - DNA, 23 bp, Artificial Sequence
  synthetic oligonucleotide primer
tgaggagggctggaacaagtacc

SEQ ID NO: 12 - DNA, 23 bp, Artificial Sequence
  synthetic oligonucleotide primer
ggaggtggtaattgcagggaaca

(19) United States
(12) Patent Application Publication
(10) Pub. No.: US 2003/0050709 A1
(43) Pub. Date: Mar. 13, 2003

(54) Trabecular bone-derived human mesenchymal stem cells

(76) Inventors: Ulrich Noth, Wurzburg (DE); Rocky S. Tuan, Chester Springs, PA (US)

Correspondence Address: David S. Resnick, NIXON PEABODY LLP, 101 Federal Street, Boston, MA, 02110, US

(21) Appl. No.: 10/082,705

(22) Filed: Feb. 25, 2002

Related U.S. Application Data
(60) Provisional application No. 60/270,977, filed on Feb. 23, 2001.

(51) Int. Cl. A61F 2/28

(52) U.S. Cl. 623/23.58; 623/23.63; 435/396

(57) ABSTRACT

The present invention discloses an in vitro engineered osteochondral graft comprising a porous matrix block, more particularly, a porous polylactic acid polymer block, press-coated with mesenchymal stem cells (MSCs), wherein a cartilage layer is formed on the surface of the matrix block. This invention may be used for treating articular cartilage defects.

### CONTINUING APPLICATION DATA

This application claims priority under 35 U.S.C. §119 based upon U.S. Provisional Patent Application No. 60/270,974 filed on Feb. 23, 2001.

### GOVERNMENT RIGHTS IN THE INVENTION

The invention was made in part with government support under grants CA 71602, AR 44501, DE 12864, AR 39740, DE 11327 and AR 45181 awarded by the National Institutes of Health. The government has certain rights to the invention.

### FIELD OF THE INVENTION

The present invention generally relates to the fields of cell biology and orthopaedic surgery and to a method of repair articular cartilage defects. More particularly, the present invention relates to an in vitro engineered osteochondral graft and the use thereof for articular cartilage repair.

### BACKGROUND OF THE INVENTION

Articular cartilage is a tough, elastic tissue that covers the ends of bones in joints and enables the bones to move smoothly over one another. When articular cartilage is damaged through injury or a lifetime of use, however, it does not heal as rapidly or effectively as other tissues in the body. Instead, the damage tends to spread, allowing the bones to rub directly against each other, thereby, resulting in pain and reduced mobility.

The repair of articular cartilage defects caused by trauma or diseases, such as, but not limited to, osteoarthritis and osteochondrosis dissecans, is a less than satisfactory process. It is a well-known phenomenon that a defect that is confined to the cartilage layer (partial defect or chondral lesion) fails to heal spontaneously. If the defect penetrates the underlying layer of subchondral bone (full thickness defect or osteochondral lesion), however, a limited spontaneous repair involving marrow progenitor cells and vascular spaces occurs; but this generally leads to the formation of less durable fibrocartilage rather than hyaline cartilage.

A number of treatment strategies for the repair of articular cartilage defects are currently in clinical use or at the experimental stage of development. Treatment strategies currently in clinical use are lavage and debridement, abrasion chondroplasty, microfracture techniques, subchondral drilling, transplantation of periosteal or perichondrial grafts, transplantation of osteochondral autografts or allografts, and autologous chondrocyte transplantation. Techniques currently at an experimental stage include the implantation of biocompatible matrices (e.g., agarose, type II collagen gels or sponges, hyaluronic acid, polylactic- or polyglycolic acid) alone or in combination with chondrocytes or growth factors, such as insulin-like growth factor (IGF) or members of the transforming growth factor superfamily-β (TGF-β).

Mesenchymal stem cells (MSCs) are cells that have the potential to differentiate into a variety of mesenchymal phenotypes by entering discrete lineage pathways. In defined culture conditions, and in the presence of specific growth factors, MSCs can differentiate into cells of mesenchymal tissues such as bone, cartilage, tendon, muscle, marrow stroma, fat, dermis and other connective tissues. These cells can be isolated and purified from a number of tissues, including, but not limited to, bone marrow, blood (including peripheral blood), periosteum, muscle, fat and dermis, and culture-expanded in an undifferentiated state in vitro. More recently, the inventor of the present invention discovered that MSCs also can be isolated from collagenase-pretreated bone fragments. This discovery is the subject matter of a co-pending U.S. patent application.

The differentiation of MSCs into cells of the chondrogenic lineage has opened new potential therapeutic approaches for the repair of articular cartilage defects. While autologous chondrocytes usually are taken from an intact articular cartilage surface, MSCs are isolated from a bone marrow aspirate of the iliac crest without a surgical procedure involving the affected joint. Also, the proliferative nature of MSCs allows them to be used as a cellular vehicle (via transfection or transplantation) to deliver gene products, such as those members of the transforming growth factor-β superfamily, to promote chondrogenesis. Furthermore, the presence of calcification in a cartilage layer restored with chondrocytes has not been observed in cartilage engineered with the use of MSCs derived from rabbits. Implantation of MSCs alone or in combination with delivery vehicles have been investigated for cartilage repair. Different matrices that have been investigated in vitro and in animal experiments as candidate delivery vehicles for MSC-based cartilage repair include, but are not limited to, collagen, hyaluronan, gelatin, or alginate gels (or composites of those). Porous bioresorbable polymers of different compositions also have been studied as delivery vehicles for MSCs. The basic approach used in these investigations is similar, i.e. loading of the delivery vehicle with MSCs.

Full-thickness cartilage defects extend into the subcondral bone. Successful articular cartilage repair requires the regeneration of the articular cartilage, subchondral bone, and integration of the repair tissue into the existing host tissue. Current approaches of implanting a delivery vehicle loaded with MSCs, however, often yield primarily bone tissue, thus failing to address this issue.

It is, therefore, an objective of the present invention to provide a method to fabricate in vitro an osteochondral graft containing a cartilage layer.

It is a further objective of the present invention to provide a method and compositions to induce regeneration of articular cartilage, subchondral bone, and integration of the repaired tissue into the existing host tissue.

### ABBREVIATIONS

“AGN” means “aggrecan”

“ALP” means “phosphatase”

“BMP” means “bone morphogenetic proteins”

“Col I” means “collagen type I”

“Col II” means “collagen type II”

“Col IX” means “collagen type IX”

“Col X” means “collagen type X”

“DBM” means “demineralized bone matrix”

“DMEM” means “Dulbecco's Modified Eagle's Medium”

“FBS” means “fetal bovine serum”

“GAPDH” means “glyceraldehyde-3-phosphate dehydrogenase”

“H/E” means “haematoxylin-eosin”

“IGF” means insulin-like growth factor.

“hMSC” means “human mesenchymal stem cells”

“hOB” means “human osteoblastic cells”

“LP” means “link protein”

“LPL” means “lipoprotein lipase”

“mhMSC” means “bone marrow-derived human mesenchymal stem cell”

“MSC” means “mesenchymal stem cells”

“OC” means “osteocalcin”

“ON” means “osteonectin”

“OP” means “osteopontin”

“PBS” means “phosphate buffered saline”

“PPARγ2” means “peroxisome proliferator-activated receptor Γ2”

“SEM” means “scanning electron microscopy”

“TGF” means “transforming growth factor”

### DEFINITIONS

“Chondrocytes”, as used herein, refers to the cells that make up the matrix of cartilage.

“Mesenchymal stem cells (MSCs)” as used herein, refers to cells that have the potential to differentiate into a variety of mesenchymal phenotypes by entering discrete lineage pathways. In defined culture conditions and in the presence of specific growth factors, MSCs can differentiate into cells of mesenchymal tissues such as bone, cartilage, tendon, muscle, marrow stroma, fat, dermis and other connective tissues. These cells can be isolated from bone marrow aspirates of the iliac crest or from other marrow containing bones and culture-expanded in an undifferentiated state in vitro.

“Chondrogenesis” as used herein, refers to the development of cartilage.

“Osteochondral grafts” as used herein, refers to transplants of tissue composed of both bone and cartilage.

“patient” as used herein, can be one of many different species, including but not limited to, mammalian, bovine, ovine, porcine, equine, rodent, and human.

### BRIEF DESCRIPTION OF THE FIGURES

FIG. 1: Representative phase contrast photomicrographs of MSCs derived from bone marrow of the femoral head. (A) MSC culture initiated from marrow cell populations consisting of red blood cells and nucleated cells. (B) Adherent MSCs after removal of the non-adherent cells at culture day 2. (C) Colony formation of hMSCs at culture day 7. (D) Confluent culture of MSCs at culture day 14. Bar=30 μm.

FIG. 2: Representative micrographs of a cell-polymer construct consisting of a 1×0.5×0.5 cm polymer block coated with 1.5×10⁶ MSCs after a culture period of 3 weeks in chondrogenic differentiation medium. (A and B) Side views of the construct showing the formation of a cartilage layer (CL) on top of the polymer. (C) Direct view onto the cartilage layer. (D) Higher magnification of A. Bars=1 mm.

FIG. 3: Representative SEM micrographs of cross-sections of the engineered cell-polymer constructs of cartilage. (A) Low magnification view of a cross-section showing the “perichondrium” on the top followed by the cartilage layer, the intermediate zone, and the acellular zone. (B) Cartilage layer lying between the “perichondrium” and the intermediate zone. (C) Cartilage layer showing cells embedded in extracellular matrix. (D) Surface of the engineered construct. Bar: (A)=150 μm, (B)=50 μm, (C)=20 μm, (D)=10μm.

FIG. 4: Histological and immunohistochemical analysis of engineered cartilage layers derived from MSCs coated onto the polymer surfaces after 3 weeks in culture. Sections were stained with H/E (4A and 4B), alcian blue (4C and 4D), picro-Sirius red (4E and 4F) or immunostained for Col II (4G and 4H), LP (4I and 4J) and Col I (4K and 4L). Asterisks denote the structure of the polymer within the cartilage layer. Arrows in Figure K and L indicate intense regions of Col I staining. Low magnification (A, C, E, G, I, and K), bar=200 μm; higher magnification (B, D, F, H, J, and L), bar=10 μm.

FIG. 5: RT-PCR analysis of the in vitro engineered osteochondral grafts (Construct) in comparison to the positive control pellets (Control) after maintenance in chondrogenic differentiation medium for 3 weeks. Shown is a representative gene expression pattern of the chondrogenic differentiation marker genes Col II, Col IX, Col X, Col XI, AGN and the expression of Col I.

### DETAILED DESCRIPTION OF THE INVENTION

The present invention relates to an in vitro engineered osteochondral graft comprising a porous matrix block and a population of mesenchymal stem cells (MSCs) prepared as high-density pellet cell cultures that are subsequently press-coated onto the top surface of the porous matrix block. Moreover, layers of morphologically distinct, chondrocyte-like cells, surrounded by a fibrous sulfated proteoglycan-rich extracellular matrix, are formed on the top surface of the porous matrix block. This engineered osteochondral graft may be implanted into a mammal for the reconstruction of partial or full-thickness articular cartilage defects. Optionally, the remaining volume of the matrix scaffold may be loaded with MSCs and/or osteoinductive growth factors prior to the implantation to elicit osteogenesis in situ and enhance osseointegration of the construct. Furthermore, the press-coating of porous matrices with MSCs also may be used in the design of in vitro engineered articular cartilage areas (e.g., medial condyle of the femur) for the restoration of an osteoarthritic joint.

Mesenchymal Stem Cells (MSCs)

The MSCs may be obtained from a number of sources, including, but not limited to, bone marrow, blood (including peripheral blood), periosteum, muscle, fat, dermis and bone by means that are well known to those skilled in the art. In one embodiment, the MSCs are obtained from bone marrow, more particularly, bone marrow aspirate of the iliac crest.

In addition to MSCs, human chondrocytes also may used in the present invention.

Porous Matrix Blocks

The porous matrix as disclosed in the present invention could be any biocompatible or biodegradable porous matrix, including, but not limited to, demineralized bone matrix (DBM), biodegradable polymers, calcium-phosphates and hydroxyapatite. In one embodiment, the porous matrix is a biodegradable polymer, more particularly, polylactic acid polymer, even more particularly, D,D-L,L-polylactic acid polymer.

The porous matrix blocks may be any shape or size that is compatible with the cartilage defect site. It is within the scope of the present invention that an osteochondral graft as disclosed in the present invention be fabricated to any shape or size prior to implantation.

Press-coating and Chondrogenic Differentiation

The process of press-coating and chondrogenic differentiation may be accomplished by 1) culturing isolated MSCs to about 70-80% confluency, 2) detaching the cells with trypsin containing EDTA, more particularly, about 0.25% trypsin containing about 1 mM EDTA, 3) centrifuging the cultured MSCs to form a high-density cell pellet, 4) gently pressing the top surface of a porous matrix block onto the high-density cell pellet in a chondrogenic differentiation medium for a first period of time sufficient enough to allow the attachment of the cells to the porous matrix block, and 5) incubating the cell-matrix construct in fresh chondrogenic differentiation medium for a second period of time sufficient enough to allow the formation of a cartilage layer.

The chondrogenic differentiation medium can be any medium that are known to those skilled in the art, that can induce the chondrogenic differentiation of MSCs. In a particular embodiment of the present invention, the chondrogenic differentiation medium contains a transforming growth factor. More particularly, in one example of the present invention, the chondrogenic differentiation medium is a serum-free, chemically defined medium that contains DMEM (BioWhittaker, Walkersville, Md.) supplemented with 10 ng/mL TGF-β1 (R&D, Minneapolis, Minn.), 100 nM dexamethasone, 50 μg/mL ascorbate 2-phosphate, 100 μg/mL sodium pyruvate, about 40 μg/mL proline and ITS-plus (Collaborative Biomedical Products, Cambridge, Mass.; final concentrations: 6.25 μg/mL bovine insulin, 6.25 μg/mL transferrin, 6.25 μg/mL selenous acid, 5.33 μg/mL linoleic acid, and 1.25 mg/mL bovine serum albumin).

In one embodiment of the present invention, the high-density cell pellet comprises about 1-2×10⁶ MSCs compressed in a pellet of about 5 mm in diameter and about 2 mm in thickness. In another embodiment, the porous matrix block is pressed on the cell pellet for about 3 hours to allow the attachment of the cells to the block, and the cell-matrix construct is incubated in a chondrogenic differentiation medium for about 3 weeks for the formation of a cartilage layer.

The amount of high-density MSCs that are required to press-coat a porous matrix block for the formation of a proper cartilage layer is determined by the size of the block and the matrix property. In one embodiment of the present invention, a high-density cell pellet of about 1.5×10⁶ MSCs is used for press-coating an about 1×0.5×0.5 cm D,D-L,L-polylactic acid polymer block, which results in a cartilage layer of about 1 to 1.5 mm. Human articular cartilage layer is several millimeters thick and rarely exceeds 3-4 mm. To increase the cartilage layer of the osteochondral graft, as disclosed in the present invention, to about 4 mm, variations of the matrix properties (e.g., pore size) or mixing MSCs with extracellular matrix proteins before coating may be applied.

Optional Loading of MSCs or Osteoinductive Growth Factors

Prior to implantation, the remaining volume of the matrix block may be loaded with MSCs and/or osteoinductive growth factors, such as bone morphogenetic protein-2 (BMP-2), to elicit osteogenesis in situ and enhance osseointegration of the implant.

Application of Engineered Osteochondral Grafts

The in vitro engineered osteochondral grafts may be used to repair articular cartilage defects by implanting the grafts to the defect site by open surgery or arthroscopy. It is preferred that MSCs from the same patient be used for the implantation. Prior to the implantation, the graft, as disclosed in the present invention, may be designed or fabricated to different sizes or shapes that are compatible to the surgery site. The present invention also may be applicable in the design of in vitro engineered articular cartilage areas (e.g., medial condyle of the femur) for the restoration of an osteoarthritic joint.

In addition, the MSCs may be genetically engineered as an effective cellular vehicle to deliver gene products, such as those members of the TGF-β superfamily to promote chondrogenesis of MSCs. Techniques for introducing foreign nucleic acid, e.g., DNA, encoding certain gene products are well known in the arts. Those techniques include, but are not limited to, calcium-phosphate-mediated transfection, DEAE-mediated transfection, microinjection, retroviral transformation, protoplast fusion, and lipofection. The genetically-engineered MSC may express the foreign nucleic acid in either a transient or long-term manner. In general, transient expression occurs when foreign DNA does not stably integrate into the chromosomal DNA of the transfected MSC. In contrast, long-term expression of foreign DNA occurs when the foreign DNA has been stably integrated into the chromosomal DNA of the transfected MSC.

Methods

Isolation and Culture of Bone Marrow-derived Human Mesenchymal Stem Cells

All chemicals were purchased from Sigma Chemicals (St. Louis, Mo.) unless stated otherwise. MhMSCs were isolated from the femoral heads of 4 patients (2 females aged 44 and 53 yr, and 2 males aged 41 and 54 yr) diagnosed with osteoarthritis and undergoing total hip arthroplasty. The cell culture procedure was modified from Haynesworth et al., Bone 13: 81,1992. Briefly, trabecular bone plugs (5-10 mL) were harvested from the cutting plane of the femoral necks using a bone curet and were transferred to 50 mL polypropylene conical tubes (Becton Dickinson, Franklin Lakes, N.J.) containing 10 mL DMEM/F-12K medium (Speciality Media, Phillipsburg, N.J.). The tubes were vortexed to disperse marrow cells from the bone plugs and centrifuged (1000 rpm for 5 min) to pellet suspended cells and bone plugs. The supernatant was discarded and the pellets were reconstituted in 10 mL complete medium consisting of DMEM/F-12K supplemented with 10% fetal bovine serum (FBS; Premium Select, Atlanta Biologicals, Ga.), antibiotics (50 I.U. penicillin/mL and 50 μg streptomycin/mL; Cellgro, Herndon, Va.), and 50 μg/mL ascorbate 2-phosphate. After vortexing, the released marrow cells were collected with 10 cc syringes fitted with 20-gauge needles and saved. The remaining cells in the bone plugs were extracted using the identical procedure for a total of five times until the bone plugs appeared yellowish-white. The collected cells were pelleted (1000 rpm for 5 min), resuspended in complete medium, counted with a hemocytometer, and plated at a density of 6×10⁷ cells per 150 cm² tissue culture flask (Corning, Cambridge, Mass.). Non-adherent cells were removed by aspiration with a pasteur pipette after 2 days and attached cells were washed twice with phosphate buffered saline (PBS). The culture medium (complete medium) was changed every 3 to 4 days.

Polymer

D,D-L,L-polylactic acid polymer blocks (OPLA®, Kensey Nash Corp., Exton, Pa.) of 1×0.5×0.5 cm were used for the coating procedure. The blocks have an apparent density (AD) of 0.0900 (+/−0.0050), void volumes of 90-92% (measured by helium pycnometry) of their apparent volumes (AV), and molecular weights (Mws) of 100,000-135,000 kDa after commercial gamma sterilization. The rate of biodegradation of the polymer is governed by multiple variables of the local tissues or culture environments. In most mammalian connective tissues OPLA® is hydrolyzed to microscopic fragments by 6-9 months and completely metabolized out of the tissue by 12 months post implantation, with faster hydrolysis in the presence of osteoinductive morphogens.

Polymer-coating and Chondropenic Differentiation

After 10 to 14 days, when the cultures reached 70-80% confluency, cells were detached with 0.25% trypsin containing 1 mM EDTA (Gibco BRL, Life Technologies, Grand Island, N.Y.) and were counted with a hemocytometer. High-density pellet cell cultures were initiated from 1.5×10⁶ MSCs in 50 mL conical tubes by centrifugation (500× g for 5 min), and formed cell pellets of 5 mm in diameter and 2 mm in thickness at the bottom of the tubes. The medium was removed and a polymer block was gently pressed onto each high-density cell pellet. To prevent the polymer from floating, the cell-polymer constructs were cultured initially in a minimal (300 μL) volume of serum-free, chemically defined chondrogenic differentiation medium. The chemically defined medium consisted of DMEM (BioWhittaker, Walkersville, Md.) supplemented with 10 ng/mL TGF-β1 (R&D, Minneapolis, Minn.), 100 nM dexamethasone, 50 μg/mL ascorbate 2-phosphate, 100 μg/mL sodium pyruvate, 40 μg/mL proline and ITS-plus (Collaborative Biomedical Products, Cambridge, Mass.; final concentrations: 6.25 μg/mL bovine insulin, 6.25 μg/mL transferrin, 6.25 μg/mL selenous acid, 5.33 μg/mL linoleic acid, and 1.25 mg/mL bovine serum albumin). After 3 hours, 2.7 mL of chemically defined medium was added to allow free floating of the cell-polymer constructs. Non-attached cells floating in the medium were removed after 24 hours when the medium was changed for the first time. The floating constructs coated with MSCs were incubated for 3 weeks at 37° C. in 5% CO₂. The chondrogenic differentiation medium was changed every 3 to 4 days. For control pellet cell cultures, 2.5×10⁵ cells were pelleted by centrifugation (500×g for 5 min) in 15 mL polypropylene conical tubes (Becton Dickinson, Franklin Lakes, N.J.) and cultured for 3 weeks in the same serum-free, chemically defined chondrogenic differentiation medium supplemented with 10 ng/mL TGF-β1

Scanning Electron Microscopy

After 3 weeks of culture the cell-polymer constructs were rinsed three times in 0.1 M cacodylate buffer (pH 7.2) and fixed overnight in cacodylate buffered 2.5% glutaraldehyde at 4° C. The specimens were post-fixed in 1% OS04 for 1.5 hr, dehydrated through a graded series of ethanol, dried in a Polaron critical point drier (VG Microtech, East Grinstead, UK), mounted onto aluminum stubs, sputter coated with gold, and viewed under a scanning electron microscope (JEOL 840, Peabody, Mass.).

Histochemical and Immunohistochemical Analysis

The cell-polymer constructs were rinsed twice with PBS, fixed for 2 hr in PBS-buffered 2% paraformaldehyde, dehydrated through a graded series of ethanol, infiltrated with isoamyl alcohol, and embedded in paraffin. Sections of 8 μm thickness were cut through the center of the constructs and were stained with haematoxylin-eosin (H/E), alcian blue, or picro-Sirius red.

For immunohistochemical analysis of Col II and LP, the monoclonal antibodies II-II6B3 to Col II and 8-A-4 to LP, obtained as ascites fluid from the Developmental Studies Hybridoma Bank, developed under the auspices of the NICHD, and maintained by the University of Iowa, Department of Biological Sciences (Iowa City, Iowa), were used. The antibodies were diluted in PBS and used at concentrations of 15 μg/mL and 6 μg/mL, respectively. Detection of Col I was done using the monoclonal antibody 1-8H5 (Oncogene Research Products, Boston, Mass.), which was diluted in PBS and used at a concentration of 1 μg/mL. Monoclonal antibody X53 to Col X (Quartett Immunodiagnostika, Berlin) was used at a 1:10 dilution. For Col II detection, sections were pre-digested with 300 U/mL hyaluronidase in 50 mM Tris (pH 8.0), 30 mM sodium acetate containing 0.5 mg/mL bovine serum albumin (BSA) and 10 mM N-ethylmaleimide for 15 min at 37° C., and incubated with the primary antibody for 1 hr at 37° C. For detection of LP, sections were digested with 1.5 U/mL chondroitinase ABC in 10 mM sodium acetate and 150 mM NaCl chloride for 15 min at 37° C. and incubated with the primary antibody overnight at 4° C. For detection of Col I and Col X, sections were pre-digested with 0.1% pepsin in 0.5 M glacial acetic acid for 2 hr at 37° C. and incubated with the primary antibody overnight at 4° C. Control groups for immunohistochemical studies were performed without primary antibodies under identical conditions. Immunostaining was detected calorimetrically using the streptavidin-peroxidase Histostain-SP Kit for DAB (Zymed Laboratories, San Francisco, Calif.). Sections stained for Col II and LP were counterstained with H/E.

RNA Isolation and RT-PCR Analysis

To ensure that all cells within the polymers were used for RT-PCR analysis, the upper half of the polymers including the coated cell layers were removed with a scalpel and total cellular RNA was isolated from the polymers or the control cell pellets using Trizol reagent (Gibco BRL, Life Technologies, Grand Island, N.Y.) and extraction with chloroform. Briefly, the polymers with the cell layer were transferred to a 1.5 mL microcentrifuge tube and dissociated in 0.5 mL Trizol using a pellet pestle (Kontes, Vineland, N.J.). RNA was extracted with chloroform, precipitated with isopropanol, and the resulting pellet stored at −80° C. in 75% ethanol. Just prior to use for RT-PCR, the RNA pellet was dried, dissolved in nuclease-free water, and the RNA concentration determined by spectrophotometry (A₂₆₀). First strand complementary DNA (cDNA) was reverse transcribed from 2 μg of total cellular RNA using random hexamers and the Superscript™ First-Strand Synthesis System for RT-PCR (Gibco BRL, Life Technologies, Grand Island, N.Y.). The amplification primers for RT-PCR as shown in Table 1 were designed and selected based on published sequences of the human Col I, (Lomri et al., Calcif. Tissue Int. 64: 394, 1999) Col II, (Su et al., Nucleic Acids Res. 17: 9473,1989) Col IX, (Muragaki et al., Eur. J. Biochem. 192: 703,1990) Col X, (Apte et al., FEBS Lett. 282: 393,1991) Col XI (Bernard et al., J. Biol. Chem. 263: 17159,1988) and AGN (Doege et al., J. Biol. Chem. 266: 894, 1991) genes. The housekeeping gene glyceraldehyde-3-phosphatase dehydrogenase (GAPDH) was included to monitor RNA loading. RT-PCR conditions were optimized by generating saturation curves of PCR products against cycle number from 15 to 40 cycles. A 2 μL aliquot of the cDNA products was amplified using a programmable Thermal Controller (MJ Research, Watertown, Mass.) in the presence of 2.5 Units Taq polymerase (Perkin Elmer, Norwalk, Conn.) at an initial denaturation for 1 min at 95° C., followed by a total of 32 cycles, each consisting of 1 min at 95° C., 1 min at 57° C. or 1 min at 51° C. (Col I), 1 min at 72° C. and a final extension at 72° C. for 10 min. DNA from 20 μL of each PCR reaction was electrophoretically separated on a 2% MetaPhor agarose gel (FMC, Rockland, Me.) containing ethidium bromide, and visualized using a Kodak Imager (Model 440 CF, Rochester, N.Y.).

TABLE 1
RT-PCR Primer Sequences and Product Size

| | | | | Product |
|---|---|---|---|---|
| | RT-PCR primer | | Position | size |
| Gene | sequences (5′-3′) | | (bp) | (bp) |
| GAPDH | GGGCTGCTTTTAAC | (SEQ. NO. 1) | 134-835 | 702 |
| | TCTGGT | | | |
| | TGGCAGGTTTTTCT | (SEQ. NO. 2) | | |
| | AGACGG | | | |
| Col II | TTTCCCAGGTCAAG | (SEQ. NO. 3) | 1341- | 377 |
| | ATGGTC | | 1717 | |
| | CTTCAGCACCTGTC | (SEQ. NO. 4) | | |
| | TCACCA | | | |
| Col IX | GGGAAAATGAAGAC | (SEQ. NO. 5) | 126-641 | 516 |
| | CTGCTGG | | | |
| | CGAAAAGGCTGCTG | (SEQ. NO. 6) | | |
| | TTTGGAGAC | | | |
| Col X | GCCCAAGAGGTGCC | (SEQ. NO. 7) | 1319- | 703 |
| | CCTGGAATAC | | 2021 | |
| | CCTGAGAAAGAGGA | (SEQ. NO. 8) | | |
| | GTGGACATAC | | | |
| Col XI | GGAAAGGACGAAGT | (SEQ. NO. 9) | 90-679 | 590 |
| | TGGTCTGC | | | |
| | CTTCTCCACGCTGA | (SEQ. NO. 10) | | |
| | TTGCTACC | | | |
| AGN | TGAGGAGGGCTGGA | (SEQ. NO. 11) | 6561- | 350 |
| | ACAAGTACC | | 6910 | |
| | GGAGGTGGTAATTG | (SEQ. NO. 12) | | |
| | CAGGGAACA | | | |

Results

Cell Culture of Bone Marrow-derived Human Mesenchymal Stem Cells

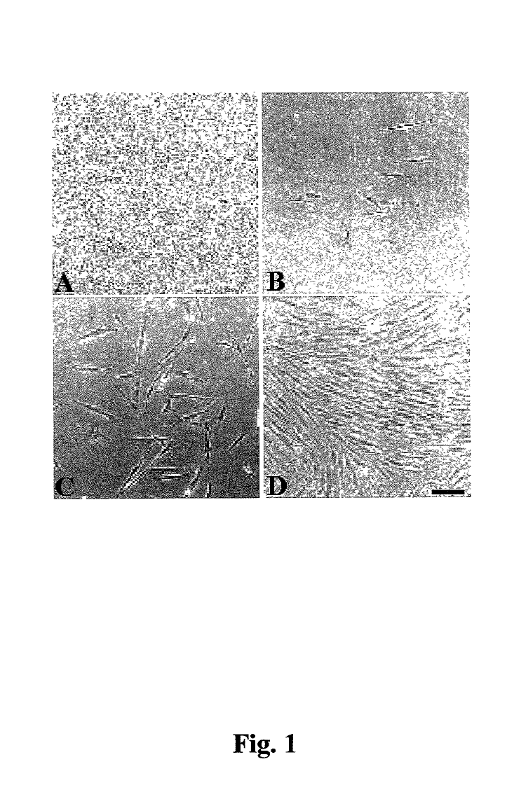

Marrow cells derived from the cutting plane of the femoral necks were plated at a density of 6×10⁷ cells per 150 cm² tissue culture flask (FIG. 1A). Five to ten 150 cm² tissue culture flasks were initiated depending on the amount of marrow cells obtained from the donor. Non-adherent cells were removed after two days by washing with medium, leaving only a small percentage of individual cells or colonies composed of a few cells attached to the plastic substrate (FIG. 1B). Typically, 500-2,000 cells remained adherent from 6×10⁷ initially plated marrow cells. No differences were found between donor age and gender. Cells replicated rapidly and formed distinct colonies within 7 days after plating, displaying a fibroblastic morphology with only a few polygonal or round cells (FIG. 1C). After approximately 14 days the cells reached confluency, retaining their fibroblastic morphology (FIG. 1D).

Polymer Coating

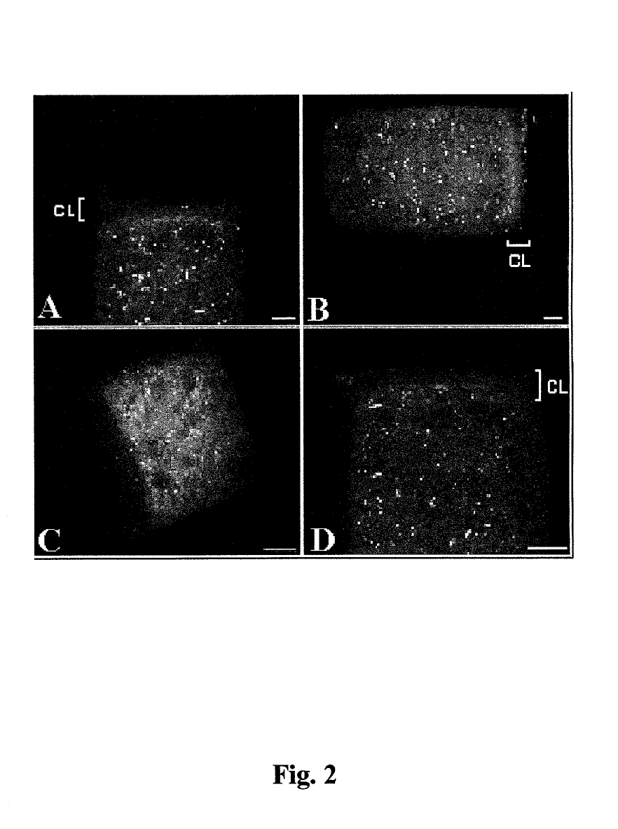

High-density pellet cell cultures initiated from centrifuged aliquots of 1.5×10⁶ MSCs formed cell pellets 5 mm in diameter and 2 mm in height at the bottom of 50 mL conical tubes. Polymer blocks of 1×0.5×0.5 cm were placed onto the cell pellets, and the cells were allowed to adhere for various times. After 3 hours most cells touching the polymer surface had attached, melding the cell pellet to the polymer block. Shorter adherence (30 minutes, 1 or 2 hours) resulted in partial attachment of the cell pellet to the polymer surface and subsequently, detachment of the pellet from the polymer occurred after the polymer construct was released to float in the medium. Initially, different seeding numbers of MSCs ranging from 0.5×10⁶ to 3×10⁶ (differing by 500,000 cells) of all donors were tested three times for the coating procedure. Coating with less than 1.5×10⁶ cells resulted in partially coated polymer surfaces. Coating with higher cell numbers resulted in overloading with the majority of the pellet coating the sides of the polymer and uneven cell layers growing on the polymer surfaces. No variation was found among different donors at any cell seeding density. At the time of harvest translucent cartilage-like layers were seen forming on top of the polymers along the originally coated surface (FIG. 2A-D). The layers appeared to be about 1 to 1.5 mm thick (FIG. 2B) without interruption along the surface but extended to different depths into the polymer depending on the surface structure of the polymer (FIG. 2C).

Scanning Electron Microscopy (SEM)

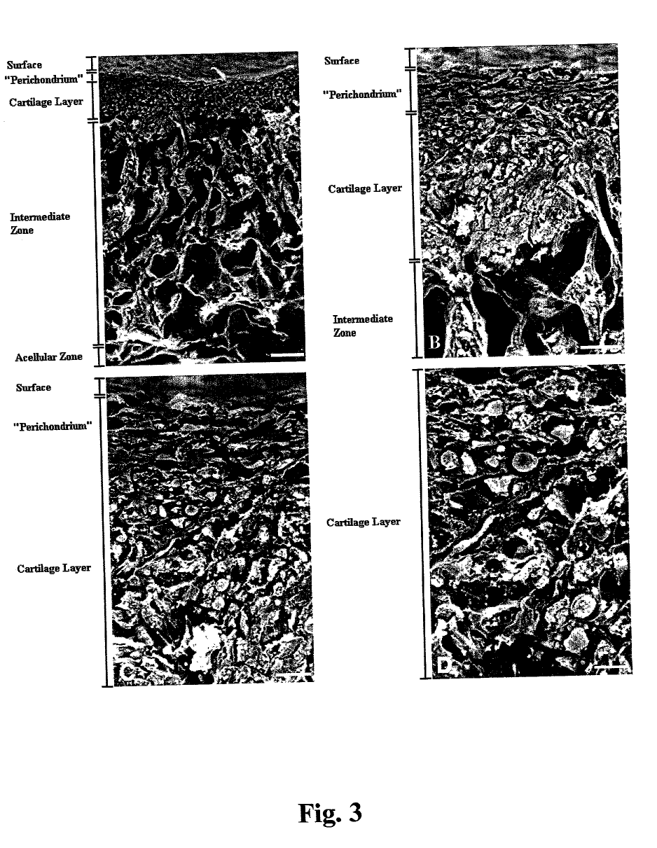

Low magnification views of sagittal cross-sections of the cell-polymer constructs revealed that the polymer surfaces were coated with a cartilage layer that varied in thickness between 1 and 1.5 mm depending on the pore indentation of the polymer at specific locations along the surface (FIG. 3A and 3B). Few elongated lining cells with little matrix production appeared as perichondrium-like cells (“Perichondrium”) at the surface of the cartilage layer (FIG. 3A and 3B). An intermediate zone where the pores of the polymer were filled with cells and extracellular matrix was located underneath the superficial cartilage layer (FIG. 3A and 3B). This intermediate zone was followed by an acellular zone where no cells could be detected within the polymer scaffold (FIG. 3A). This acellular zone was typically located about 1 to 1.5 mm from the surface. Higher magnification of the cartilage layer showed chondrocyte-like cells embedded in abundant extracellular matrix (FIG. 3C). Views of the surface of the engineered constructs showed an uninterrupted superficial cell layer (FIG. 3D).

Histochemical and Immunohistochemical Analysis

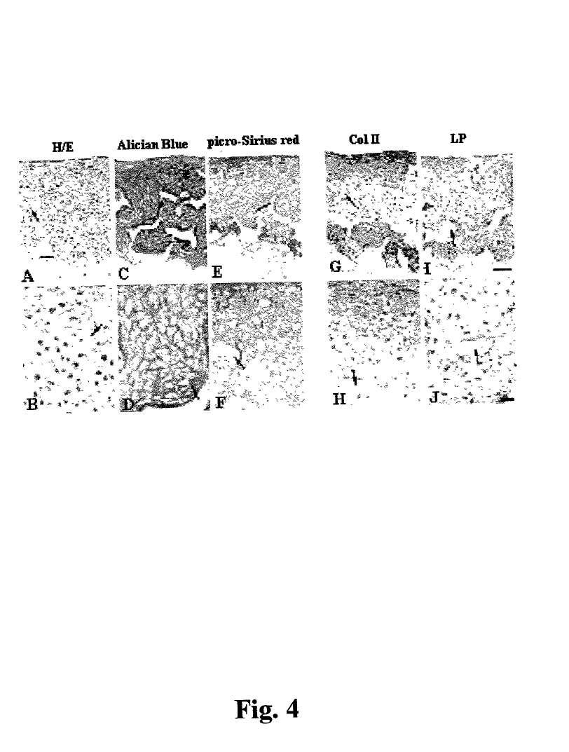

Sections of the cell-polymer constructs maintained in the chondrogenic differentiation medium for 3 weeks and stained with H/E showed morphologically distinct, round chondrocyte-like cells embedded in extracellular matrix (FIG. 4A and 4B). Staining with alcian blue revealed the presence of a negatively charged sulfated proteoglycan-rich extracellular matrix (FIG. 4C and 4D),and staining with picro-Sirius red showed prominent orange-red birefringent fibers in the matrix and surrounding the cells (FIG. 4E and 4F). Immunostaining of the cell-polymer sections detected the presence of Col II predominantly at the outer and inner part of the cartilage layer, while the middle part stained less intense (FIG. 4G and 4H). LP was detected throughout the cartilage layer with most intense staining at the inner part (FIG. 4I and 4J). Col I staining was highest at the outer perichondrium-like surface and the surfaces facing the polymer embedded in the cartilage layer (FIG. 4K and 4L). On the other hand, no detectable Col X immunostaining was observed in these cell-polymer constructs (data not shown).

RT-PCR Analysis

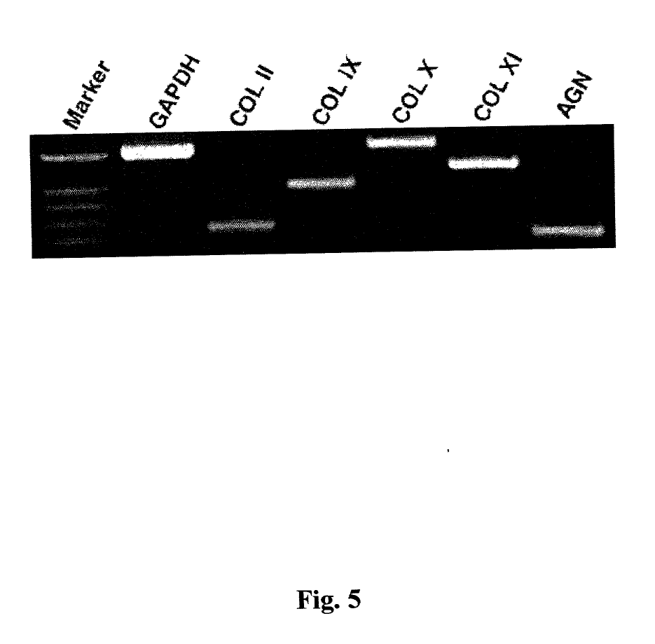

Total RNA was isolated from the cartilage layer bonded to the polymer and the positive control cell pellets (cultured without polymer) after 3 weeks of culture. RT-PCR analysis revealed the mRNA expression of the chondrogenic marker genes Col II, Col IX, Col X, Col XI, and AGN by the engineered constructs. Expression of Col I also was found (FIG. 5, lower panel).

RT-PCR analysis was carried out for two independent constructs generated from all patients and the results were similar. The gene expression profile resembled that of positive control cell pellets cultured without polymer (FIG. 5, upper panel). Cartilage constructs and positive control cell pellets generated from the different donors showed the same gene expression pattern.

Discussion

The present invention discloses the development of in vitro engineered cell-polymer constructs formed by press-coating biodegradable polymers with MSCs for use in the reconstruction of articular cartilage defects. The technique involves the utilization of MSCs prepared as high-density pellet cell cultures that are subsequently press-coated onto the surface of porous biodegradable polymer blocks.

As a basis for the constructs D,D-L,L-polylactic acid polymer blocks, that have been optimized for architectural compatibility with cancellous bone were used. This commercially available biodegradable polymer is in clinical use as a support matrix for bone remodeling in maxillo-facial surgery and has been well characterized regarding its physical and chemical properties and its biological compatibility. The polymer also has been used successfully as a delivery vehicle for BMP-2 in bone tissue engineering of critical size defects in the rabbit radius ostectomy model. As a cell source, MSCs isolated from the femoral head of patients undergoing total hip arthroplasty were used. The surgical waste nature of the femoral head obviates the need for more complicated patient consent agreements, generally required for marrow aspirates from the iliac crest, the more common source of MSCs.

The invention presented herein shows that these cells are able to undergo chondrogenesis under defined culture conditions as previously described for MSCs derived from the iliac crest. Articular cartilage is a relatively acelluar tissue with an extracellular space occupied by interstitial fluid (60-80%) and organic extracellular matrix components, primarily Col II and proteoglycans. Immunohistochemical analysis of sections of human articular cartilage have shown that Col II is uniformly distributed within the cartilage matrix, while Col I is found in the subchondral bone, the periosteum, the perichondrium, the cytoplasm of hypertrophic and degenerative chondrocytes, and in the matrix of fibrocartilage. The immunohistochemical analysis of sections of the engineered osteochondral graft detected Col I predominantly at the surface of the construct and the surfaces facing the polymer embedded in the cartilage layer, suggesting the formation of fibrous, perichondrium-like layers at these regions. Because Col I also was found within the cartilage layer, the phenotype of the engineered cartilage cannot be strictly defined as articular cartilage. Nevertheless, Col II, a typical marker of hyaline cartilage could be detected histochemically and by RT-PCR.

Besides Col II, articular cartilage also contains small amounts of other collagens such as collagen types V, VI, IX, X, and XI. The exact function of these minor collagens is yet not fully understood. The engineered cartilage layer showed mRNA expression of Col IX, which has been found at the surface of the Col II fibril and may be involved in mediating fibrillogenesis via collagen-collagen or collagen-proteoglycan interactions. Also detected was Col XI mRNA, which is attributed to cartilage collagen and controls cartilage collagen fibril formation.

Recently, it has been shown that MSCs cultured as high-density pellets and maintained in chondrogenic differentiation medium supplemented with TGF-β3 can be further differentiated to the hypertrophic state by addition of thyroxine, the withdrawal of TGF-β3, and the reduction of the dexamethasone concentration. Hypertrophic cartilage is found in the growth plate of fetal and juvenile long bones, ribs, and vertebrae and contains a short-chain collagen, Col X, which is unique to this tissue and is only found elsewhere under pathological conditions, e.g., in osteoarthritic articular cartilage and in chondrosarcoma. Col X gene expression could be shown by the sensitive RT-PCR technique but no protein was detected by immunostaining, suggesting little or no Col X production by the engineered cartilage layer. In fact, considering that the generation of a functional osseochondral junction is desirable for articular cartilage re-surfacing, low amounts of Col X, normally associated with hypertrophic chondrocytes, may be advantageous for proper tissue integration.

Proteoglycans form a special class of glycoproteins with attached highly charged glycosaminoglycans, which are strongly hydrophilic and dominate the physical properties of the proteoglycan. While cartilage has a high proteoglycan content (5-7%), bone matrix is predominantly mineral with a low proteoglycan content (0.1%). Aggrecan, which is not present in bone, is the major proteoglycan in cartilage, and is important for expanding and hydrating the extracellular matrix. Link protein strengthens the aggrecan-hyaluronan bond by forming a ternary complex in the matrix. Both aggrecan and link protein were detected within the engineered cartilage layer indicating secretion of these proteoglycans by the chondrocytes.

Human articular cartilage is several millimeters thick and rarely exceeds 3-4 millimeters. Cartilage of the high weightbearing joints of the lower limb is thicker compared to the upper limb with variations of the thickness within each joint. The cartilage layer engineered in the present invention showed a thickness of up to 1.5 mm as revealed by histological analysis and SEM. Therefore, the fabricated layer is about half the thickness of human articular cartilage of the high weightbearing joints of the lower limb.

The present invention provides a new method for the development of in vitro engineered cell-polymer constructs coated with MSCs that is useful for the reconstruction of partial or full-thickness articular cartilage defects. The required MSCs may be isolated from a bone marrow aspirate of the iliac crest or femoral head and used for the in vitro design and fabrication of cartilage layers of different sizes and shapes. Prior to implantation, the remaining volume of the polymer scaffold may be loaded with MSCs and/or osteoinductive growth factors (e.g., BMP-2) to elicit osteogenesis in situ and enhance osseointegration of the construct. Furthermore, press-coating of polymers with MSCs also might be applicable in the design of in vitro engineered articular cartilage areas (e.g., medial condyle of the femur) for the restoration of an osteoarthritic joint.

While this invention has been described with a reference to specific embodiments, it will obvious to those of ordinary skill in the art that variations in these methods and compositions may be used and that it is intended that the invention may be practiced otherwise than as specifically described herein. Accordingly, this invention includes all modifications encompassed within the spirit and scope of the invention as defined by the claims.

What is claimed is:

1. An engineered osteochondral graft for promoting the growth of cartilage in a patient at a defect site in need of repair, comprising a matrix block and a first population of MSCs, wherein said first population of MSCs are press-coated on a top surface of said matrix block, and said first population of MSCs forms a cartilage layer on said top surface of said matrix block.

2. The engineered osteochondral graft of claim 1, wherein said matrix is biodegradable.

3. The engineered osteochondral graft of claim 2, wherein said matrix is selected from the group consisting of demineralized bone matrix (DBM), biodegradable polymers, calcium-phosphates and hydroxyapatite.

4. The engineered osteochondral graft of claim 3, wherein said matrix is a porous polylactic acid.

5. The engineered osteochondral graft of claim 4, wherein said porous polylactic acid is D,D-L,L-polylactic acid.

6. The engineered osteochondral graft of claim 5, wherein said matrix block is a D,D-L,L-polylactic acid polymer block of about 1×0.5×0.5 cm, said top surface of said matrix block is about 0.25 cm², said first population of MSCs is about 1.5×10⁶, and said cartilage layer is about 1-1.5 mm thick.

7. The engineered osteochondral graft of claim 1, wherein said matrix block has a shape compatible with said defect site.

8. The engineered osteochondral grafted of claim 1, wherein said MSCs are isolated from a tissue selected from the group consisting of bone marrow, blood, periosteum, muscle, fat, bone and dermis.

9. The engineered osteochondral grafted of claim 8, wherein said MSCs are isolated from bone marrow.

10. The engineered osteochondral graft of claim 1, wherein said engineered osteochondral graft further comprises an osteoinductive growth factor in an amount sufficient enough to elicit osseointegration.

11. The engineered osteochondral graft of claim 10, wherein said osteoinductive growth factor is BMP-2.

12. The engineered osteochondral graft of claim 1, wherein said engineered osteochondral graft further comprises a second population of MSCs which are loaded in the remaining volume of said matrix block, and said second population of MSCs is in an amount sufficient enough to elicit osseointegration.

13. The engineered osteochondral graft of claim 12, wherein said engineered osteochondral graft further comprises an osteoinductive growth factor in an amount sufficient to elicit osseointegration.

14. The engineered osteochondral graft of claim 13, wherein said osteoinductive growth factor is BMP-2.

15. The engineered osteochondral graft of claim 1, wherein said first population of MSCs are transiently or stably genetically engineered to express a gene product.

16. The engineered osteochondral graft of claim 15, wherein said gene product is a member of the transforming growth factor-β superfamily.

17. A method of fabricating an osteochondral graft comprising the steps of contacting a top surface of a matrix block with a high-density pellet of a population of MSCs for a first period of time sufficient enough to form a cell-matrix structure, and culturing said cell-matrix structure in a chondrogenic differentiation medium for a second period of time sufficient enough to form a cartilage layer on said top surface of said matrix block, wherein said population of MSCs is an amount enough for the formation of said cartilage layer.

18. The method of claim 17, wherein said chondrogenic differentiation medium contains a transforming growth factor.

19. The method of claim 18, wherein said transforming growth factor is a member of TGF-β superfamily.

20. The method of claim 19, wherein said member of TGF-β superfamily is selected from the group consisting of TGF-β1, TGF-β3 and BMP-2.

21. The method of claim 17, wherein said first population of MSCs is about 1.5×10⁶ cells per 0.25 cm² of said top surface area.

22. The method of claim 17, wherein said matrix block is a D,D-L,L-polylactic acid polymer block of about 1×0.5×0.5 cm, said top surface is about 0.25 cm², said population of MSCs is about 1.5×10⁶, said first period of time is about 3 hours, said second period of time is about 3 weeks, and said chondrogenic differentiation medium contains about 10 ng/ml TGF-β1.

23. A method of promoting the growth of cartilage in a patient at a site in need of repair, comprising the step of implanting an engineered osteochondral graft at said site, wherein said engineered osteochondral graft comprises a matrix block and a first population of MSCs, wherein said first population of MSCs are press-coated on a top surface of said matrix block, and said first population of MSCs forms a cartilage layer on said top surface of said matrix block.

24. The method of claim 23, wherein said engineered osteochondral graft further comprises an osteoinductive growth factor in an amount sufficient enough to elicit osseointegration.

25. The method of claim 23, wherein said engineered osteochondral graft further comprises a second population of MSCs which are loaded in the remaining volume of said matrix block, wherein said second population of MSCs is in an amount sufficient enough to elicit osseointegration.

26. The method of claim 25, wherein said engineered osteochondral graft further comprises an osteoinductive growth factor in an amount sufficient to elicit osseointegration.

27. The method of claim 23, wherein said first population of MSCs are transiently or stably genetically engineered to express a gene product.

28. The method of claim 27, wherein said gene product is a member of the transforming growth factor-β superfamily.
